(19) United States
(12) Patent Application Publication
(10) Pub. No.: US 2014/0358044 A1
(43) Pub. Date: Dec. 4, 2014

(54) Normothermic maintenance system and method

(71) Applicants: Laurence Anthony Kirwan, Norwalk, CT (US); Stephen John McLouglin, Aspe Heath (GB)

(72) Inventors: Laurence Anthony Kirwan, Norwalk, CT (US); Stephen John McLouglin, Aspe Heath (GB)

(21) Appl. No.: 13/986,724

(22) Filed: May 29, 2013

(51) Int. Cl. A61F 7/02; A61H 99/00

(52) U.S. Cl. CPC A61F 7/02; A61H 99/00; A61F 2007/0233
USPC 601/18; 607/112

(57) ABSTRACT

A normothermic maintenance system sand method for maintaining patient normothermia, during surgical intervention is disclosed. The invention will reduce patient morbidity during surgical intervention while beneficially expediting patient flow in the hospital environment. The application of heat, via advantageously independent systems and methods, is an objective of the invention which results in the improvement of the economics of surgical treatment by the use of either disposable or re-usable simple normothermic maintenance systems.

The instant invention relates generally to surgery and a method, apparatus, and system for maintaining normothermic conditions in a patient before, during and after surgery.

### BACKGROUND

The instant invention relates to methods and systems for maintaining patient normothermia, particularly during surgical intervention. One objective of the invention is to reduce patient morbidity during surgical intervention while beneficially expediting patient flow in the hospital environment. Yet another objective is the application of heat via advantageously independent systems and methods. Yet a further objective is the improvement of the economics of surgical treatment by the use of either disposable or re-usable simple normothermic maintenance systems.

The inventors believe that prior art in this domain fails to adequately address the specific practicalities of the surgical environment which failure significantly contributes to the development of patient hypothermia. The inventors intend that the instant method and system acts to maintain normothermia rather than allowing patient hypothermia to establish itself and then seek to redress the hypothermic condition. The inventors further consider and will show that heating the extremities rather than heating the torso is a more practical solution for the prevention of hypothermia.

Maintain normothermia in the patient rather than correct for hypothermia

Reduce morbidity through maintenance of normothermia

Improve operating room environment

Intervention-less garment

Application of heat to the extremities.

In the field of surgery it is recognized that maintaining the patient at a euthermic temperature has both a therapeutic and a comfort value. Historically the therapeutic value largely went unrecognized and the comfort value of the patient was subordinated to the comfort of the surgeon, presumably on the basis that the patient was unconscious.

What is now recognized is that application of hypothermia as a deliberate element of the surgical procedure should be restricted to zonal surgeries where particularly sensitive tissue damage may result from normal or elevated corporeal temperatures such as in cardiac surgery or neurosurgery.

Christensen et al U.S. 2011/0172749 define and confirm a traditional perspective on hypothermia and the inter-relationships between different elements of the circulatory system: “Hypothermia . . . is the result of prolonged exposure to a cold challenge where blood flow through the venous plexuses and AVA's can be near zero of the total cardiac output. Vasoconstriction of the peripheral blood vessels may arise under hypothermia in order to prevent further heat loss by limiting blood flow to the extremities and reducing heat transfer away from the thermal core of the body. However, vasoconstriction makes it much more difficult to reverse a hypothermic state since vasoconstriction impedes the transfer of heat from the body surface to the thermal core and makes it difficult to simply apply heat to the surface of the body. This physiological impediment to heat transfer is referred to as a vasoconstrictive blockage to heat exchange.”

The instant inventors, agree with the Christensen physiological analysis but contend that the effects of psychological stress is an additional, un-quantified significant, yet understated, contributor to the early onset of hypothermia in the surgical environment.

In the surgical environment, the initial stages of hypothermia start to occur in the pre-operative period when the patient is asked to disrobe; this procedure typically occurs in a cool environment. The patient is then given a surgical garment. Surgical garments currently in use are not conducive to insulating the body, or to the maintenance of normal body temperature. They are often skimpy, poorly fitting, garments, open at the back, with minimal coverage of the patient's extremities and severely compromise the patient's modesty and self-respect: all of which contributes to the patient's anxiety and heightens sensitivity to external stimuli in an unfamiliar environment. The patient is then left in an alien environment, partially clothed and potentially hypothermic which again results in a relatively high level of anxiety. The resultant increases to pulse rate, cardiac output and increasing secretion of adrenergic stress hormones such as adrenaline, cortisol and insulin are characteristics of a ‘fight and flight’ physiological response. It is well known that adrenaline is a potent vasoconstrictor and that peripheral vaso-constriction and redirection to the core is a standard physiological response to stress, which further aggravates the ability of the body to maintain a homeothermic environment and therefore predisposes to hypothermia.

Practically, this physiological response creates difficulties in inserting intra-venous (IV) cannulas in the ambulatory/pre-surgical holding area as well as compounding the difficulties of efficient monitoring of oxygen saturation in the operating room with application of pulse oximeters to the digits.

The inventors conclude that these environmental and physiological factors contribute to normothermic degradation prior to anaesthesia, while significantly increasing the difficulties of patient care.

The inventors therefore conclude that early stage hypothermia is being induced in the pre-operative process of preparing the patient for surgery in the presurgical holding area and the operating theatre and that the negative impacts of the propensity to early onset of the hypothermic condition have not been fully appreciated.

In addition, incremental developments in surgical technique, driven by technological advances are changing the timing of patient throughput. Writing in the Journal of Anaesthesia 23(2), 2009: pp 230-4, Sasano et al report that significant increases in the time progression from the patient's operating room entrance to incision are being observed. These time delays are attributed to recent advances in surgical technology. The study reported that the time taken in 2008-2009 was 30.4 minutes (+/−8.8 minutes) longer than the time for similar procedures in 1985-1986. Such delays are almost certain to have a significant negative impact on the patient's body temperature, health and wellbeing and the inventors seek to address this through the use of their invention.

The nominal temperature of the human body should be 37° C. with hypothermia being defined as a core temperature <36.0° C., although the U.S. Army define the upper threshold of hypothermia as being a core temperature of <35° C., possibly in response to the age and fitness of their corps of personnel. A normal and desirable core temperature state is referred to as ‘normothermia’. The condition of hypothermia is further sub-divided into mild, (32-35° C.), moderate, (26-31° C.) and deep (18-25° C.). Augustine et al 2002/0183813 [Abandoned Application?] confirms normothermic temperature limits for the body as being 37° C. +/−1° C. further teaching “ . . . surface tissue varies in temperature according to whereon the body it is located. The skin of the torso is usually hypothermic, while the skin of the legs is always hypothermic. The normal skin temperature of the distal leg of a patient is approximately 32° C., which is considered to be “moderately hypothermic”. The skin temperature of the distal leg of a patient with vascular insufficiency may be as low as 25° C., which is “severely hypothermic.” Augustine confirms the necessity for maintaining normothermia: “This extremely tight temperature control is necessary because virtually all cellular functions, chemical reactions and enzymatic reactions are optimum at normothermia”.

Industry literature widely acknowledges that, except in cases where tissue degeneration is deliberately arrested by cooling, hypothermia is generally detrimental to patient recovery and may put life at risk and this is reflected in a large number of learned publications.

A study by Karalapillai et al, (2011) published in Anaesthesia 66(9): 780-784, September 2011, which utilized an extensive dataset derived from surgical interventions conducted in Australia and New Zealand sought to correlate surgical risk and hypothermia and additionally to create definitions for transient and persistent hypothermia. The study was able to successfully correlate increased risk of death in elective cardiac surgery patients with persistent hypothermia. “Persistent hypothermia” was defined as a core temperature of less than 36° C. which endured for in excess of 24 hours. Karalapillai comments: “Transient hypothermia was not independently associated with increased hospital mortality (OR=0.9, 95% Cl 0.8-1.1), whereas persistent hypothermia was associated with markedly increased risk of death (OR=6.3, 95% Cl 3.3-12.0).” The inventors note that persistent hypothermia as defined by Karalapillai et al is only 1° C. lower than normothermic temperature.

Another study by Qadan et al, entitled “Hypothermia and Surgery: immunologic mechanisms for current practice and published in Annals of Surgery 250(1): 130-140, July, 2009, commenced, “Anesthetized surgical patients are particularly at risk for hypothermia, which has been directly linked to the development of sequelae, such as coagulopathy, infection, morbid myocardial events, and death after surgery,” they were further able to form the conclusion: “Hypothermia exerts multiple effects at the cellular level, which impair innate immune function, and are associated with increased septic complications and mortality.”

In a paper entitled “Effects of perioperative hypothermia and warming in surgical practice”, published in International Wound Journal, 2(3): 193-204, (2005) Kumar et al, reported: “Perioperative hypothermia is common and adversely affects clinical outcomes due to its effect on a range of homeostatic functions. Many of these adverse consequences are preventable by the use of warming techniques . . . . The primary beneficial effects of warming are mediated through increased blood flow and oxygen tension at tissue level. Reduction in wound infection, blood loss and perioperative pain with warming is promising . . . . Achieving normothermia throughout the patient's journey is a worthwhile goal in surgical patients.”

In addition to the clinical benefits of removing hypothermia, Defina et al in the Journal of PeriAnesthesia Nursing 13(4):229-235, August, 1998, noted that the removal of hypothermia was observed to result in improvements in the economics of patient health, concluding; “The inadvertent hypothermia that is often seen after anesthesia in a cool environment has been associated with delays in recovery and longer stays in the PACU . . . . Patients who arrived in the PACU hypothermic had longer PACU stays than patients who arrived normothermic.” Need definition of PACU

A decade later a further study was able to put statistics to Defina et al's conclusions. Writing in the periodical Best Practice and Research Clinical Anaethesiology 22(4): 645-657, December 2008, Reynolds et al reported: “Only a 1.9° C. core hypothermia triples the incidence of surgical wound infection following colon resection and increases the duration of hospitalization by 20%. Furthermore mild hypothermia triples the incidence of postoperative adverse myocardial events. Thus, even mild hypothermia contributes significantly to patient care costs and needs to be avoided.” Clearly, in addition to patient welfare, there are significant financial implications to removal of patient hypothermia.

The benefits of sustained normothermia in surgical patients have the gross effect of reducing the morbidity rate of surgical intervention. Predictable reduction of patient morbidity enhances patient wellbeing and patient throughput can thereby be increased with significant benefit to both patient and surgical centers. In summary, the effect of hypothermia on surgical patients is particularly extreme: the peri-operative environment of pre-surgical areas, operating rooms and post anaesthesia care units are typically maintained at lower than normal ambient temperatures. Traditional medical surgical garments fail to offer the necessary insulative construction or material properties to be able to maintain normothermic body temperature in this environment.

Finally, the process of anaesthesia, which reduces metabolic rate, further conspires to reduce body temperature.

The autonomic defense reflex of shivering, which could at least temporarily or partially alleviate the hypothermic condition and by which the body indicates a requirement to increase core temperature is not available to the anaesthetized patient. Shivering generates between two and three times the normal metabolic thermal production. The patient's inability to shiver therefore serves to further reduce body core temperature.

The effect of hypothermia extends beyond the immediate surgical environment. In an article, entitled, “Antibiotic prophylaxis in clean surgery: clean non-implant wounds” by Leaper et al, published in Journal of Chemotherapy, 13 Spec No 1 (1): 96-101, 2001 November, the correlation was drawn between reduced requirement for antibiotic prophylaxis and the maintenance of normative temperature during surgery, concluding: “Wound infection after clean surgery (the majority being hernia, varicose vein and breast surgery) is often greatly underestimated. If a trained and blinded observer is involved using close and prolonged surveillance . . . , an infection rate of 15% or more may be found. Equally controversial is the value of prophylactic antibiotics in preventing postoperative wound infection; there is no clear cut evidence of efficacy and some random controlled trials have shown no differences at all . . . . An alternative to antibiotics is the systemic warming of patients or the local warming of the operative site prior to surgery.”

Peri-operative heating of the patient is clearly considered advantageous. However, prior art in this field has created conflict between the practical application of existing garments and blankets and peri-operative surgical procedures which detrimental combination conspires to reduce the efficacy of the heating process and simultaneously creates significant levels of additional complexity in the surgical environment. Summarizing; existing heat devices conflict with the requirements of the surgical environment.

Further academic studies have been conducted which consider the negative impact which intra-operative heating devices have on the surgical environment and to determine if pre-operative heating alone is sufficient to remove surgically induced hypothermia. These studies concluded that pre-surgical heating is insufficient and that the patient derives optimal benefit if artificial heat is also applied during the intra-operative and post-operative phases.

It is therefore the inventors' intention to improve peri-operative warming and coincidentally create, through the use of their invention an improved environment for the surgical team. Coincidentally and as a further benefit to both patient and surgical team, the maintenance of normothermia will, additionally, reduce unwanted fluctuations in pulse rate, cardiac output and create an improved homeostatic platform and promote efficient wound perfusion.

Existing devices preferentially consist of garments and blankets which focus heat on the torso. However, the instant method and device focuses on the prevention of hypothermia through the provision of heat via essentially tubiform garments which encase the limbs and extremities.

Additionally, the majority of existing heat devices are tethered to the patient and to fixed points located in the surgical environment. These heating blankets and garments require removal prior to surgery, and additionally at one or more times during surgery. For example, in the pre-surgical environment, the surgical site is marked by the operating surgeon thereby necessitating the heat garment removal. The inventors observe that the removal and replacement of existing surgical warming devices creates opportunity for the patient to become hypothermic while requiring additional, undesirable, time-consuming and costly intervention by skilled nursing staff and they seek to eliminate this unnecessary element in the surgical procedure in order to maintain patient normothermia.

Making a direct comparison with existing technologies, when deploying the instant invention, for preference, the patient clothes themselves in the instant device(s) in the pre-surgical environment and the devices thereafter require little additional intervention by nursing or medical-surgical personnel, thereby reducing the number of tasks to be completed by the surgical staff and simplifying the patient-care algorithm. This procedural advantage continues for the duration of the surgical procedure and confers significant physiological, operational and economic benefits over prior art.

As the devices proposed by the instant invention are advantageously disposed on the patient's limbs, this leaves the primary surgical area of the torso free and unencumbered. The proposed method simultaneously and advantageously provides heat to a larger proportion of the body surface area, “BSA”, than is possible with existing systems and devices.

The torso represents only 32% of the human Body Surface Area, “BSA”, and is an area which incorporates significant thermal insulation in the form of fat and tissue layers. The extremities however, have a higher ratio of BSA to body mass/volume, which correlates with increased sensitivity to ambient temperature and accelerated heat loss. More importantly, the limbs represent a larger element of the human Body Surface Area, (“BSA”). If the entirety of the limbs is covered with the instant invention up to 58% of the body surface area can be covered: this represents an increase in area of approximately 80% over existing methods and technologies. Heating the patient's limbs, therefore, should be favorably compared and contrasted with heating the patient's torso. Therefore, by quantitatively treating a greater skin surface area and qualitatively applying heat to an area of greater thermal sensitivity, heat transfer to the limbs acts collaboratively to provide significant improvements to patient normothermia.

As a large percentage of the major organs which require surgical intervention are located on the torso, deployment of the instant invention does not interfere with the surgical site. Recent statistics from the American Society of Plastic Surgeons indicate that 36% of surgeries conducted in 2011 were conducted exclusively on the torso. This figure rises to 49% with the inclusion of non-specified liposuction. Thoracic and/or abdominal surgical operations typically allow only a portion of the torso to be covered during the procedure, which therefore reduces the effectiveness of existing heating devices. Furthermore, blanket type devices tend to apply zoned heating such that in the zones which are removed or de-activated there is, effectively, no patient heating and therefore significant potential for areas of the patient's torso to be subjected to significant heat loss. Such heat blankets also leave the patient's arms uncovered, thereby contributing to further increases in heat loss. Additionally, blanket type devices provide unwanted heat and noise to the environment of the operating room, to the detriment of the surgical staff. On occasion existing torso-heating devices may be supplemented by additional cover for the arms, or for the legs, thereby increasing the effective coverage area. However, these devices are typically inflatable which increases the bulk of the patient in the surgical environment, encroaching on the available space in the operating room and creating significant difficulties for the surgical team.

The instant invention allows the active heating device to be worn in proximity to the skin surface as a garment which does not require additional securing to the patient. Advantageously the instant device reduces complexity in the surgical environment as it does not require connection via by an umbilical attachment to an external heat and electrical source.

Turning now to the existing surgical environment, the majority of surgical devices deployed at present which promote normothermia require ancillary equipment. In one embodiment these take the form of hot air blowers which have the tendency to increase the ambient air temperature, to the detriment of the surgical staff. Such devices are also connected by umbilical conduits which serve to connect the patient to external heat or electrical sources. The umbilicals themselves create additional, unwanted complexity in the surgical environment and pose significant, attendant Health & Safety Hazards. Other embodiments which promote normothermia by heat transfer from circulating hot liquids around garments also disadvantageously require umbilicals and additional tethering. Therefore, yet another advantage of the instant device is that it does not require additional ancillary equipment which equipment may adversely impact the surgical environment.

Finally, prior art in this field includes devices which transmit heat by convection and radiation. The instant device more advantageously transmits heat to the patient predominantly by conduction and radiation.

Existing methodologies for patient peri-operative heating provide for intermittent heating: the pre-operative garments are removed for patient marking as mandated in the Joint Commission National Patient Safety Goals Jan. 1 2012, UP001.02.01 and then exchanged for intra-operative garments or detached from their heat source for transport to the operating room. These physical and temporal windows, typically of at least 10 minutes duration, leave the patient's torso without thermal protection and therefore subject to considerable detrimental heat loss.

In addition and when deploying existing heating methods and systems, surgical procedures conducted on the torso frequently leave only a portion of the torso actively heated, or the lower extremities alone, thereby reducing the efficacy of the heating device. The instant invention, however, allows surgery to be conducted on the torso while continuous active heating is being applied throughout the preoperative, intra-operative and postoperative phases and most significantly, without interruption.

The inventors maintain that by encasing the limbs in un-tethered active thermal devices in the pre-surgical area, that vasoconstriction is reduced or eliminated, peripheral blood flow maintained and that there is, significantly, no interruption to the provision of heat to the patient, either during the marking up procedure, or for the duration of the surgical procedure. Cardiac output, respiratory rate, heart rate, oxygenation and levels of circulating insulin, cortisol, adrenaline and inflammatory enzymes such as cytokines, are thereby maintained within the normal range.

This is compared with current methodologies where the heating garments may be changed as many as four times throughout the peri-operative period. The inventors deduce that despite their application, existing systems and methods for maintaining normothermia often contribute to intermittent hypothermia resulting in sustained detrimental reduction in core and peripheral temperatures which the body has difficulty reversing. The inventors therefore conclude that existing systems are economically wasteful and propose a method and system which does not require inter-operative intervention nor result in unnecessary wastage.

In summary, prior art in the field of surgical heating, has been to provide thermal heating via the torso, and occasionally provide heat to the lower extremities during surgery. The logic supporting this appears to be founded upon existing practices for the treatment of hypothermia. Typically, for outpatients, the treatment of hypothermia is a matter of emergency treatment. The symptoms of hypothermia are proportional to the degree of body cooling and vary between mildly hypothermic; shivering, lethargy, ‘clammy’ skin and hyperventilation and the severely hypothermic state, where symptoms may include unconsciousness, dilated pupils, decreased respiratory rate and ultimately cardiac dysrhythmias and asystole. As body heat has already been removed from the extremities and the locus of remaining body heat is in the torso, the treatment for hypothermia consists of two stages; arresting the hypothermic condition by increasing the core temperature of the body, then once the core temperature is restored, the body self-regulates temperature to the extremities. Custom and practice show that the correct procedure for the emergency treatment of hypothermia is the application of heat to the torso.

The inventors contend that correcting for hypothermia and maintaining normothermia present different practical anatomical challenges and thus propose an alternative method and system of heating the patient for the maintenance of the normothermic condition.

The inventors further conclude that existing methods, systems and apparatus conceived for the prevention of surgically induced hypothermia have been constructed following the rationale used for the treatment for existing hypothermia, in that prior art in this field largely concentrates on heating the torso. The inventors further consider and will show that heating the extremities is a more practical solution for the prevention of hypothermia.

Existing devices for the correction of surgical hypothermia can largely be categorized into those applying heat directly to the patient and environmental thermal control devices seeking to control the patient's environment, such as heated under-blankets or operating tables which have integrated heat sources. Without exception, existing peri-operative patient thermal systems and devices require intervention, fitting, adjustment and in some cases, calibration in the operating room. The inventors perceive that inadvertent harm may befall the patient as a result of the protracted delay in applying heating. In addition, the requirement to fit existing devices in situ detracts from the workflow of the operating room. It is the inventors' intention to address this defect.

Of the existing systems and apparatus which apply heat directly, the majority are tethered devices which convey heat from an external device to the patient via an umbilical. The inventors observe that tethered devices contribute significantly to increasing the complexity of the operating room. Additionally, in a study reported in the Canadian Journal of Surgery 53(3): 189-195, June 2010, by Wong et al report that the most common cause of delay in the operating room was attributable to equipment failure. Although clearly there are many types of surgical equipment, the inventors consider that by removing electro-mechanical heating devices, their invention contributes to making the operating room a less cluttered, simpler, less failure prone environment and therefore a more effective and productive workplace.

Numerically it would appear that the majority of the existing intellectual property in this domain pertains to “Forced Air Warming” or “FAW” devices. There are some inventions, however, which are stand-alone in that the heat source is contained within the garment or blanket. The form of the direct heat application devices also varies widely from blankets to garments which are loosely configured about the torso.

Prior art does not, however, encompass the application of heating to the patient's limbs. Garment apparatus may therefore be further sub-categorized into whole body garments covering the torso and garments applied to the extremities.

Prior art also includes a wide variety of different heating modes. These include electric heating elements, forced air warming devices, heat packs, fluid circulation, chemical heating and the like. The subsequent discussion of prior art further serves to confirm the significant advantages of the instant invention.

Anderson et al, U.S. Pat. No. 7,914,566 “Multifunction warming device with provision for warming hands” acknowledge the importance of keeping the patient's hands warm, disclosing a convective warming blanket form garment with slits in the sides through which the patient's hands may be warmed by the operation of a forced air warming convective apparatus. This patent represents an improvement over Anderson et al.'s prior expansive intellectual property which is disclosed therein and which includes U.S. Pat. No. 7,364,584 Multifunction warming device with provision for warming hands. Anderson's device, while apparently increasing the versatility of forced air warming devices may also unfortunately impede patient monitoring as pulse oximetry devices typically require access to the fingers, which access may be impeded by the patient's hands being enclosed within the inflated garment. The potential difficulties in deploying such devices in the operating theatre are acknowledged in Anderson et al.'s later evolutionary intellectual property, U.S. Pat. No. 8,097,031 Warming device with provisions for deploying elements of an upper body convective apparatus and for deploying the lower portion of the warming device, which acknowledges the importance of being able to heat a portion of the patient's torso, the heated portion being independent of the site of surgical intervention.

Recent intellectual property to Panser et al, U.S. Pat. No. 8,070,797 and of common ownership with Anderson, discloses a refined clinical garment which applies heat via a forced air warming device [“FAW”] which is partitioned such that different heat modes may be applied to different parts of the body. Unlike the Anderson intellectual property, U.S. Pat. No. 7,364,584, U.S. Pat. No. 7,914,566 and U.S. Pat. No. 8,097,031 the modular construction of the Panser garment allows for sleeves to be attached to the garment in order to apply heat to the arms. Collectively the Anderson and Panser garments seek to create environmental partitions enabling differentiation between comfort and therapeutic warming. Although this increases the versatility of the heat emitter, it requires a dual mode forced air warming device machine which is attached via bulky umbilicals and which significantly leaves more of the body surface area, (BSA), unheated than is practically heated. Additionally, such devices create significant undesirable environmental impact in the operating theatre.

Surgical, patient and environmental control devices may have undesirable bi-products in the form of increasing the heat and noise of the operating room environment.

Noise levels created by the hot air blower are a distraction to the surgical team and frequently result in requests to disconnect the power in procedures of short duration or in ones in which only a small portion of the patient's body surface area (BSA) is exposed, e.g. facial, foot or hand surgery. This also reflects poorly on the efficacy and cost effectiveness of these devices.

In addition, Albrecht et al, reporting in the American Journal of Infection Control 39(4), 321-328, in May, 2011 concluded that incorrect selection or inadequate installation procedures on the air filters used on Forced Air Warming devices can lead to increased levels of airborne microbial contaminants. Additionally, they concluded that there was little evidence that the efficiency of the air intake filter was adequate to protect the internal air path from buildup of microbial contamination within the machinery of the FAW. This, then, requires the FAW to be serviced by a technician, further adding to the cost and service personnel requirements of the operating room. The instant invention has no such requirement, thereby representing a simplification over prior art.

Forced Air Warming, “FAW”, devices, due to their inflatable nature, are inherently unstable so that a cotton blanket is almost invariably applied over the device. Perversely, the blanket is maintained in a warming closet and also requires recycling thereby further adding to procedural costs. The instant device solves this problem by being in garment form, by not requiring securing to the patient and by being conformably disposed about the patient's body. It can be applied in the forms of sleeves, trousers or a shirt type garment for ease of application in cases where it will not impede or infringe on the surgical field. As the umbilical heating tube from the FAW device to the inflatable garment is hidden beneath a blanket and surgical drapes it cannot be monitored in order to verify the effectiveness of the distal connection to the patient's garment. Nor is there any alarm to alert the operating personnel in the event of disconnection. In this event, the unmonitored loss of effective warming is a further draw-back of existing FAW devices. The instant invention has no such drawbacks.

Paolini et al, U.S. 2006/0162085, “Inflatable Blanket with a tie” propose a refinement over existing intellectual property in that the forced air warming blanket is equipped with integral tie-down straps. The straps serve a dual function, namely the control of the blanket position and also the degree of inflation of the device on the patient's torso. Paolini et al confirm the problematic nature of securing an inflatable device to a patient in such a way as to be secure during surgical interventions. The instant invention dispenses with tie-downs.

Augustine et al, U.S. 2008/0103567, U.S. Pat. No. 8,062,343, “Heating blanket”, disclosing yet another variation on forced air warming blankets acknowledges the advantages of warming the patient's “head and at least one arm, allowing the patient's chest and abdomen [to] remain substantially exposed.” The patent states, “arms tend to be excellent heat exchange surfaces”. A further benefit claimed by Augustine is securing the heating blanket to the patient such that the blanket remains stationery relative to the patient's body position. The instant invention concurs with Augustine on the subject of heat transfer in the limbs and claims the advantage of simplicity over Augustine of being a garment, which is advantageous in that being a garment it does not require attachment to the patient and furthermore, is incapable of slippage or displacement into the sterile surgical field.

The instant invention therefore represents advances over prior art to Anderson, Augustine, Panser, and Paolini et al in that it creates a normo-thermative environment for the patient without creating detrimental heat and noise in the surgical environment. Significantly it has a lower capital cost and is preferably disposable, although a re-usable version is equally within the scope of the instant invention. Additionally, it does not require a static multi-patient device which requires constant servicing and electrical certification by trained biomedical engineers, which further adds to per-case cost as well as fixed facility overhead. Unlike blanket devices, it does not require attaching to the patient during surgery either by tethering or by a heat transfer umbilical as it is in self-contained garment form. Furthermore it is worn for the duration of the entire surgical encounter comprising the pre-surgical, surgical and post-surgical phases, which results in minimal temperature fluctuations and minimal pre-surgical thermal loss to the patient. In addition it applies heat to the limbs which is advantageous to the circulatory system.

Another embodiment of a heating device patented by Vergona et al US 2009/0299442 Warming blankets covers, and apparatus, and methods of fabricating and using the same, disclose warming blankets with pockets mounted therein, wherein each of the one or more pockets is adapted to hold a respective removable warming element. Vergona's patent specification affirms that the first two hours of surgery result in the highest degree of thermal loss per hour and proposes that the blanket will start to generate heat within 20 minutes of exposing the heat packs to the air, and continue to generate heat for approximately 12 hours which is within the time taken for the majority of surgeries. Personal observation by Kirwan further narrows the onset of hypothermia to the first 10-15 minutes of the procedure. In this time interval, the patient is completely disrobed for purposes of marking, to apply Intermittent Compression Stockings, urinary catheters, FAW devices and to prepare and drape the patient, prior to making the initial incision. The interval between when the patient enters the operating room and first incision is always recorded and can vary from 10-30 minutes, at the end of which the patient's core temperature is typically 35° C. Partial restoration of the core temperature over time is possible using FAW devices, but the hypothermia largely remains uncorrected for the duration of the surgery.

Vergona's device further proposes that it is maintained in place purely by its own weight. The removable warming elements by which Vergona proposes heating the patient, consist of chemical compositions which, when activated by exposure to air have an exothermic reaction. This device has some advantageous design elements, in particular its lack of requirement to be attached by an umbilical, but, being a blanket it still has the potential to impinge on or contaminate the sterile surgical field and has limited application in the pre-surgical, ambulatory, holding area.

The invention exposed by Vergona et al does not, unlike the instant device, propose zonal focusing of the heat requirement. It is the intention of the instant invention to apply heat in a focused manner, preferentially in zonal areas of the patient's body where maximal heat absorption is assured.

Harris et al, Adjustable Disposable Surgical Thermal Blanket, U.S. 2006/0178717 teach a layered blanket containing a plurality of discrete, sealed compartments containing solid, particulate oxygen activated exothermic composition with seals defining the compartments. Harris claims advantage over Vergona in that it is disposable, additionally requiring minimal surgical staff intervention.

The invention proposed by Harris anticipates the requirement for access to the patient during surgery by permitting partial or complete detachment of one or more of the compartments to allow reconfiguration of the heating device in accordance with the particular surgical requirements pertaining to a particular operation. Additionally Harris claims pre-operative, intra-operative or post-operative blanket configuration. This device, like all of the blanket devices, has placement issues, it being positionally difficult to control during surgery. Additionally, having sealed pockets requires the activated pockets to be accurately placed with respect to the surgical area. However, the blanket is unnecessarily wasteful in that the detachable areas are disposed of without being used, thereby unnecessarily increasing total unit cost.

The instant invention represents an improvement over Harris in that it is worn by the patient, rather than being in blanket form. Additionally it targets the most advantageous zones for the introduction of heat to the patient's circulatory system, which represents the most beneficial and direct application of heat, and thereby requiring fewer active heating elements with which to achieve comparable heating results which has the added benefit of reducing net unit costs. It also approaches the problem of hypothermia in a more logical physiological way by maintaining normothermia and avoiding the onset of hypothermia at any time.

A second group of patents proposes preferentially heating the extremities. Salmon et al, 2003/0040783, Warming Apparatus, disclose a rigid, non-conformable re-usable apparatus designed to apply radiant heat energy to a patient's hand, which advantageously uses areas of high concentrations of arteriovenous anastomoses (“AVA”) to ensure maximal heat absorption. Salmon also claims the advantage of included pulse-oximetry sensors; however, Salmon fails to address the issue of IV access for administration of intravenous fluids. In addition, detrimentally, Salmon proposes the application of heat to only one hand which the inventors assert is insufficient to combat hypothermic onset.

Although Salmon proposed the application of heat to a single limb, the active heating device enclosing the arm is required to be tethered to a base unit via an umbilical. Therefore, if the patient requires IV cannulas, the practical inference is that the IV catheter will have to be inserted into the opposite arm which is also in use for the Blood Pressure Cuff, thus leaving the lower extremity for intra-venous, “IV” access. This therefore requires the tethered attachment of both of the patient's arms and a lower extremity to external loci, which has detrimental impact on the surgical environment.

The instant inventors consider that although applying heat to one extremity is somewhat beneficial, by applying heat to multiple limbs and by maintaining the heating device as a self-contained, stand-alone unit benefits both the patient and the surgical environment. Practically, the instant invention claims advantage in that it allows for application of a cannula to the dorsum of either hand which is being heated and an independent pulse oximeter to the index finger of the same hand, thus creating greater flexibility and versatility in the surgical environment.

The instant invention also claims additional advantage over Salmon in economic terms by not requiring technician intervention and by preferentially being constructed for single use and therefore disposable. Alternate configurations of the instant invention may be constructed to have disposable heat elements disposed in pockets and to allow for re-cycling the garment after removal of the spent heat elements and appropriate sterilization of the garment.

Christensen et al, in a patent entitled Methods and Apparatus for Enhancing Vascular Access in an Appendage to enhance Therapeutic and Interventional Procedures disclose a tethered device “for increasing blood flow, controlling the vasodilatation of a patient's vascular structure, regulating the temperature of a portion of a mammal, and for improving various interventional procedures and/or therapeutic techniques.” The invention is, in effect, an electro mechanical heater, umbilically coupled to a sleeve into which the patient's limb is inserted. The sleeve controls body temperature by circulating warm water there through and also incorporates temperature measurement and feedback mechanisms and which device may also preferentially exert vacuum pressure to further improve circulation.

As with Christensen, the instant invention preferentially utilizes one or more of the patient's limbs as a means of transferring heat to the patient, but without the disadvantages of having an external power device, or of requiring significant surgical staff intervention, or of being attached by an umbilical to the patient which attachment is both inconvenient and as previously disclosed acts to encroach on the surgical environment. The instant device therefore claims benefit of simplicity over Christensen.

Furthermore the proposed invention claims advantage through the ability to compensate for obesity in patients. Taking, as a starting point the Lund-Browder burn chart, and applying new methods for compensating for obese patients as proposed by Neaman et al in a research paper published in May/June 2011, in the Journal of Burn Care & Research, and used in conjunction with the instant invention, even a morbidly obese patient can receive active heating to 50% of their BSA, which represents a significant advantage over existing technologies and systems.

In addition, the method proposes that applying heat to the limbs is more efficacious due to the superior heat dissipation and absorption mechanics of arteriovenous anastomoses, “AVA's” located in the extremities of mammalian limbs.

There are a number of non surgically oriented patents which have as their subject matter the application of heat to the limbs and in particular the hands. These devices are typically for use by sportspeople and people who are exposed to the outdoor environment, although some devices incorporate structural support mechanisms for therapeutic usage. One particular invention to Helenick, U.S. Pat. No. 6,141,801 concerns a combined therapeutic heat and support glove which heats the dorsum of the hand. Helenick also discloses gloves which are designed to combat cold temperatures and which are designed for outdoor activities. With few exceptions these devices focus the active heat element on the dorsum of the hand, leaving the palm of the hand and fingers free to be tactile. The instant invention claims advantageous use of the area of arteriovenous anastomoses which are located on the palm of the hand in order to more effectively apply heat to the patient, while additionally claiming the economic benefit of disposability.

Rinehart, U.S. Pat. No. 5,035,003 teaches the use of a glove with a bladder and an external, self-contained heat source, the objective being to create a convective circulatory system within the liquid filled bladder from which to transfer heat to the fingers. The instant invention claims advantage over Rinehart by virtue of simplicity in that there is no liquid filled bladder and also by economic advantage of simplicity of construction and disposability which is advantageous in the surgical environment.

Walasek et al, U.S. Pat. No. 5,050,596 “Reusable and microwaveable hot or cold therapy mitt and method of manufacture” disclose a multi-layered device which can be wrapped and tethered about the patient's extremities as required. The instant invention claims advantage over Walasek in that it is self-sustaining as it does not require a microwave or external power source to activate it. This makes the instant invention readily adaptable for field or emergency use where there are no external power supplies. Furthermore, the instant invention is pre-formed and close fitting about the patient's extremities, which format advantageously allows for effective heat transfer between the heat element and the patient's extremities. Where the effectiveness of heat transfer in Walasek is dependent on the skill of the person applying the heat garment to the patient, the instant device requires no external support or assistance in order to arrive at optimal heating, resulting in a more easily controlled application of heat to the patient.

In a more recently awarded patent, U.S. Pat. No. 7,043,768 to Gogarty, reveals a thermal gel filled glove. The glove is heated and largely disposed about the wearer's hand. Although designed for therapeutic treatment, the device would prove problematic in the surgical environment as the gel filled elements preclude intra-venous catheterization. The instant invention claims the advantage of simplicity, improved economics and utility over Gogarty as the heat transfer element is integral to the glove which heat dissipation mechanism allows for heat transference across a larger body surface area.

Finally, Haensel, U.S. Pat. No. 6,268,595 Circulation Warmer, proposes an apparatus and method for warming the blood of a user prior to distribution to an extremity of a user which includes a source of energy suitable for distribution as heat. Haensel's device incorporates transfer paths, heat positioners and energy state changes, which represent a considerable degree of complexity. Such complexity is inconsistent with the requirements placed upon a normothermic device by the demands of the surgical environment and therefore, the instant invention claims the benefit of simplicity over Haensel as the device requires no integral energy state changes and additionally there are no fluid transfer paths incorporated therein.

### SUMMARY OF THE INVENTION

The instant invention pertains to the method of maintaining normative body temperature in surgical patients in the peri-surgical environment utilizing a self-contained heat supply garment.

The device takes the form of a passively insulative garment to which integral heat-elements are added. The garment clothes the patient's extremities and is worn by the patient throughout all phases of the surgical encounter. The patient therefore benefits peri-surgically from an uninterrupted application of heat with which to maintain normothermic body temperature.

The instant garment is worn such that it beneficially targets areas of the patient's physiology where there is an inherently high thermal transfer efficiency thereby maximizing heat transfer from the device to the patient. These areas are typically located on the limbs. Once clothed in the garment heat-elements are activated, providing up to 12 hours of heat to the patient, which time period is sufficient to accomplish the majority of surgical interventions. Heat is dissipated within the garment by conductive and radiative means, via a tracery or weblike network of heat-conductive elements and convectively via optional heat channels located proximate the patient's dermis. The heat elements are preferably in indirect contact with the patient's skin with heat loss from the elements being minimized through insulative external covers. Furthermore, thermal inks marked onto the external surface of the garment and physically connected to the area of the garment proximate the patient's dermis via heat conductive elements may be used to provide surgical staff with simple visual confirmation of garment functionality.

A further physiological benefit, derived from the maintenance of normothermic temperature, is the avoidance of peripheral vasoconstriction. This greatly facilitates the insertion of an IV cannula in the pre-operative phase as well as aiding the monitoring of oxygenation via pulse oximetry during surgery. Therefore the instant invention suggests additional advantage of applying heat mechanisms to the limbs as it offers greater opportunities for problem-free provision of intra-venous access.

Therefore, in summary, the instant method and device provides a superior method of providing heat to a surgical patient while simultaneously providing for increased surgical access and minimizing the detrimental effect on the environment of the operating room.

### BRIEF DESCRIPTION OF THE DRAWINGS

FIG. 1: is a timeline schematic of the patient's core temperature taken in relation to peri-operative procedures, showing the benefits of the instant method relative to existing devices and methodologies. Also taken into consideration is an hypothetical example of skin temperature taken from the lower extremities.

FIGS. 2a through 2c: show isometric drawings of a partial section of the instant device for deployment on a limb

FIG. 3: shows a cross sectional view of the device as configured for a nonspecific limb, particularly illustrating the heat elements, heat dispersive elements and insulative configurations.

FIGS. 4a, 4b and 4c: show cutaway perspectives of the upper torso, illustrating several alternative configurations of the garment for deployment on the patient's upper extremities and incorporating detail of the glove configuration.

FIG. 5 shows an embodiment of the device as deployed on the lower limbs of the patient:

FIGS. 6a, 6b and 6c: illustrates isometric partial cutaway dorsal and ventral drawings of the device as configured for deployment on both the lower and upper limbs

### DETAILED DESCRIPTION OF THE PREFERRED EMBODIMENTS

It will be readily understood by those practiced in the art that the components of the instant invention as generally described and illustrated herein can be designed and arranged in a variety of different configurations. Thus, the accompanying more detailed descriptions of the embodiments of the system and method of the present invention are in no way intended to limit the scope of the invention. Additionally, those skilled in the art will appreciate that various modifications to the detailed schematic diagrams contained herein may be made within the scope of the invention without departing from the essential characteristics of the invention as described herein.

The instant device comprises a method, system and apparatus pertaining to the deployment and disposition of a disposable or substantially re-usable heating garment preferably configured for application to the patient's limb or limbs.

The method of the instant device in the pre-surgical environment consists, of the patient disrobing and clothing themselves with the garment to one or, preferentially, more than one limb. This represents an advantage over existing methods and technologies in that the patient's core body temperature does not have time to be deleteriously affected prior to entering the operating room. A further advantage of the instant device is that the patient dresses themselves, removing the requirement for intervention by surgical staff. This helps to preserve patient modesty and avoids any risk of liability arising from inappropriate actions on the part of the attendant staff.

Therefore, at a minimum, the patient encases one extremity or limb within the confines of the device. Dressing more than one limb is considered advantageous as it reduces the skin area of the body which is exposed to cool air which is prevalent in operating rooms. Simultaneously the patient benefits from the application of active heat via particularly advantageous skin forms. In its most beneficial format where all four limbs are enrobed, 58% of the total Body Skin Area, (BSA) can simultaneously benefit from a combination of active heat application and passive insulation.

This should be compared and contrasted with existing surgical devices which seek to insulate and heat the torso. Assuming that a surgical procedure allows for the anterior and posterior of the entire torso to be heated, the resultant area for applying heat and insulation is 34%. However, this figure assumes the application of heat to both front and back of the torso. Typically, the application of FAW devices is restricted to the anterior aspect of the body and, occasionally the anterior aspect of the extremities. As a consequence of this, if surgery is to be performed on the anterior torso of the patient, the area of total coverage may suffer further reduction, resulting in an active heating area of 16% or less of the BSA.

By comparison, even if the instant device is only applied circumferentially to the lower limbs and extremities of the arms, i.e. the wrists and hands, the resultant total area of the body which is heated and insulated would be 44% of the total body surface area. This ability to apply heat to a larger percentage of the BSA, without impinging on the site of surgery, therefore, represents a significant improvement over prior art.

In order for the instant device to provide less Body Surface Area insulative coverage it would have to be applied to only one arm or one leg, the inference being that the area being occupied by the operation would be the remaining arm, leg and the entirety of the torso, which seems an unlikely combination for a single surgical intervention.

Even with only a single limb being encased, the advantageous skin areas being treated still create significant advantages over prior art. The inventors also perceive that their invention may be successfully and advantageously deployed in multi-site surgical interventions such as liposuction. Thus the instant invention preferentially provides the potential to apply both active heat distribution and passive insulation to a larger proportion of the patient's body surface area than has hitherto been achievable in prior art in order to achieve the invention's objective of maintenance of normothermic patient temperature.

In addition to the normothermic benefits conferred to the wearer of the instant device the inventors consider that the method of deployment of their invention will serve to reduce the incidence of patient morbidity as, unlike existing devices which seek to recover the patient's core temperature, the instant method and apparatus acts to prevent the onset of hypothermia during pre-operative preparation. Patient morbidity consists of a range of sequelae which may include deep vein thrombosis, hematoma, infection, and cardiac dysrhythmias and ischemia.

The inventors also consider that, in line with The Joint Commission National Patient Safety Goals, NPSG.03.05.01, Jan. 1, 2012, the instant invention may have significant benefit in reducing the requirement for anticoagulation therapy. The Commission noted that; “Anticoagulation medications are more likely than others to cause harm due to complex dosing, insufficient monitoring, and inconsistent patient compliance.” The inventors note that the prevention of hypothermia has significant benefit in terms of improving patient circulation and preventing the formation of blood clots, particularly in the extremities. As has been previously remarked, having active heating deployed during the pre-surgical procedures where, traditionally, the patient's torso is uncovered and subject to significant heat loss, acts to prevent the onset of hypothermia.

Notwithstanding the several benefits conferred by active heating of the limbs, use of the instant invention, advantageously, does not preclude the advantageous deployment of passive heating garments to the rest of the patient's torso.

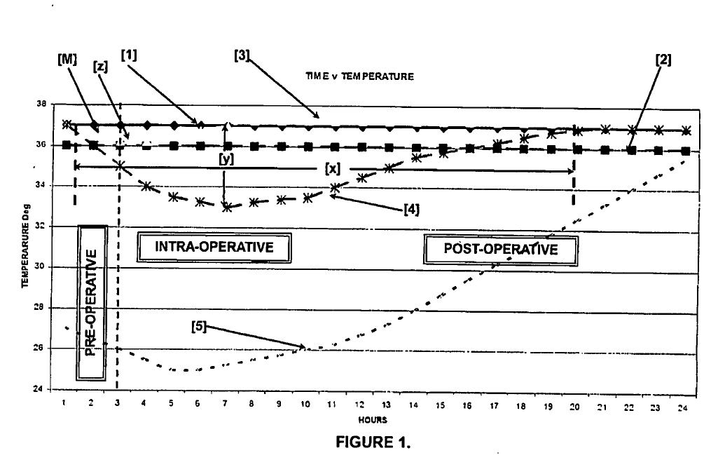

Turning now to FIG. 1 which illustrates a hypothetical timeline with associated temperature curves and which serves to illustrate the beneficial method of the instant device. The normative baseline core temperature of a human body is represented as a horizontal line at the 37° C. meridian 1.

Standard pre-operative medical procedures typically result in reduction to core temperature. The anticipated average heat loss during this pre-surgical period is between 1.5° C. and 2.0° C. 4. Further thermal degradation of the core temperature is exhibited during the “marking-up” and surgical preparation, [M] when the patient's torso is left substantially uncovered. On initiation of the surgical procedure, the body is typically equipped with heating means, which enables partial recovery and prevents further degradation of core temperature during the surgical procedure—illustrated by temperature curve 4 Note that there is a prolonged delta temperature offset between the normal and the partially recovered temperature, (x). Whereas the temperature offset between normative core temperature 1 and recovered temperature may be relatively small, the same cannot be said to be true for the offset temperature between core and the extremities, where the skin temperature may be as much as 12° C. lower than the core temperature.

As an example of core temperature recovery using FAW, an initial, theoretical patient lower limb temperature, with a negative 4° C. offset and initial, ideal temperature of 37° C. has been selected 4. For a patient with impaired circulation, utilizing existing medical devices may result in a temperature decay curve to the extremities which mimic the core temperature decay, 5. This thermal decay is only restored through the application of FAW or other external heat sources; the patient is incapable of restoring core temperature without assistance. The resultant delta offset prior to correction of the core temperature through provision of heat from a conventional heat source is labeled as (y). The primary reason that the delta offset never closes with baseline core temperature is the result of inefficiencies in the traditional method of convective heat application and the relatively low percentage of the BSA to which the heat is being applied.

By comparison, it is the intention of the existing heat method to maintain normothermic core temperature and enhanced skin temperature to the extremities by applying conductive heat across a larger and in particular a more receptive surface area.

Although, as an indicator of the stability of patient health, it is clearly important to maintain body core temperature, in terms of reducing patient morbidity it may be equally significant to reduce the offset between core temperature and the skin temperature of the extremities.

As the dynamics of heat loss relate the magnitude of heat loss to the temperature offset between two adjacent elements, increasing the temperature of the patient's extremities to mitigate heat loss from the core may ultimately prove to be as important to patient wellbeing as maintaining core normothermic temperatures. A typical reduction in base temperature, after the application of heat to the torso is illustrated at “y” and the proposed offset temperature range after heating the extremities is illustrated at “z”. What is evident is that the greater the temperature offset between core and limbs, the larger the potential for heat loss from the body core and the steeper the patient's temperature decay curve.

Turning now to the construction of the garment 10, specific physical characteristics demand the use of materials with particular properties which promote and sustain garment functionality. The garment 10 should be constructed to simultaneously confer both thermally insulative and thermally dispersive attributes. It is envisaged that this may require properties which are outside the scope of a single fabric type and a laminar construction may be envisaged in order to confer all the necessary physical attributes. Optimal functionality may also require different active and passive garment characteristics to be applied to different parts of the patient's enclosed limbs. As an example, the lamellar construction of the garment enclosing the patient's hand may not be the same as for the patient's forearm or arm.

In a further advantageous embodiment, the garment may be tailored such that the heat output may be customized to meet the specific requirement of an individual. In this way, heat distribution may be advantageously managed such that not only is the core temperature 1 normalized, but the temperature offset [z] between extremities and core temperature may also be optimized.

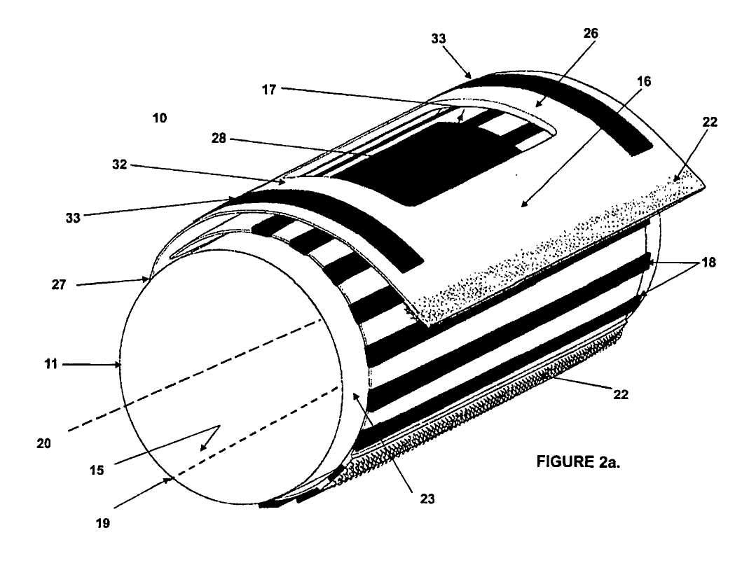

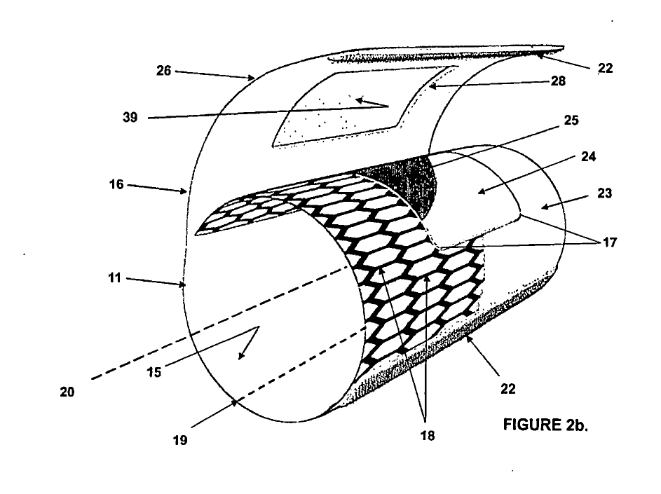

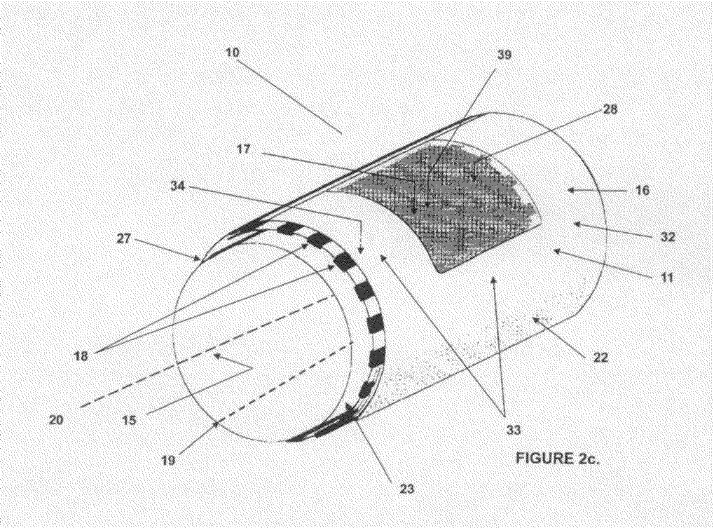

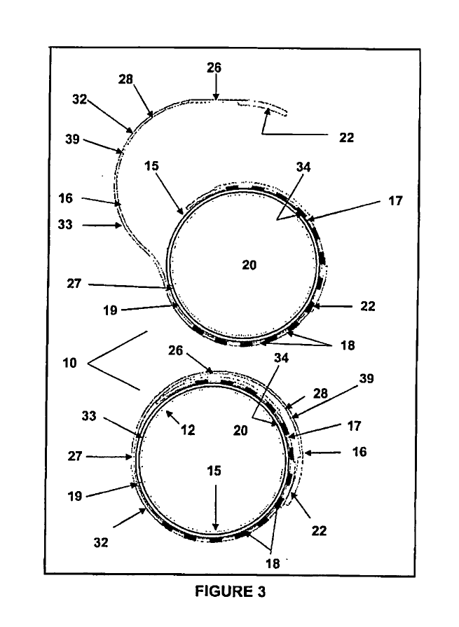

FIGS. 2a, 2b and 2c, therefore, represent simple isometric cross sections which consider alternative embodiments of the instant device and FIG. 3 is a simple cross section of the instant device 10. The figures represent the instant invention as deployed and arranged on a (non-specific) human limb 20

Starting with the fabric layer in closest proximity to the limb, the material, 11 whether paper or fabric or any other form of flexible or pliable material is selected from a group of materials which have the required physical and thermal properties. The fabric may be of a class of fabrics which are manufactured in essentially continuous reels of tubiform materials or, alternatively, may be constructed from rolls of fabric, stitched, glued, welded or formed in any other manner, as required.

The fabric layer 15 proximate the patient's skin 12 may preferentially be reinforced or thickened to convey additional insulative properties at particular, predetermined and specific parts of the garment 10 in order to accomplish the goal of efficient distribution and retention of heat energy to the patient's extremities. The characteristics of this inner layer of material 15 are selected from a range of properties which promote comfort, passive warming, and heat dissipative and insulative properties. Furthermore, by fitting the garment 10 in proximity to the dermis 12, air-flow between the patient's skin and the device 10 can be controlled, thus enhancing the thermal conductivity and insulative properties of the device 10. For this reason the fabric surface closest the dermis 12 may be profiled or textured to channel warm air in proximity to the patient's skin 12.

Additionally, the inner fabric layer 15 may be perforated to allow convection of the warm air which is trapped between the inner fabric layer 15 and the outer covering 16 of the device 7. In a further beneficial arrangement, the innermost insulative fabric layer 15 may be deployed beneath the heat pack 17 in order to mitigate heat concentration and maintain the heat supply at a desired skin temperature of T<41.5 C. If temperatures in excess of 40° C. are applied directly to human skin over a significant time period, skin may be damaged by excessive heat concentration. The lower threshold for heat damage to skin due to heat is reported and defined by Moritz and Henrique (1947) as either 44° C. or 45° C. More recent works, in particular that of Xu and Qian (1995) predict the time threshold for a first degree burn to be 16 hours at a skin temperature of 42° C. and 6.5 hours at a skin temperature of 43° C. Clearly, therefore, the insulative layer 15 proximate the patient's skin 12 should be constructed so as to maintain the resultant exothermic temperature between 40.5° C. and 41.5° C.

The material selected for the construction of the inner fabric layer should have physical properties which relate the heat output of the selected exothermic device 17, measured in Watts, to the surface area of the heat pack 17, measured in cm2. The resultant energy output is therefore measurable in W/m2. Heat diffusion mechanisms can then be constructed which are sized so as to be responsive to the patient's specific thermal requirements. For example, a patient who has poor circulation may have a greater peri-operative and intra surgical heat requirement and the garment may require less passive insulation between the skin and the active heating element in order to attain normothermia. In an alternative configuration, a garment or garments for clothing the same patient may be constructed with normal levels of passive insulation and a larger distributive network of heat conductive filaments 18 in order to more effectively disperse heat to the patient. In other words the passive and active elements of the instant design work collaboratively to create the appropriate degree of heating in order to maintain patient normothermacy.

The essentially tubiform garment 10 is equipped with a detachable “seam” 19 which preferentially runs along the principal axis of the patient's limbs 20. This seam 19 is positioned such that, in a surgical emergency, the entire garment 10 can be removed instantaneously and without recourse to equipment of any sort. The seam element 19 may be continuous or discontinuous in nature. The seam may be constructed in such a manner that the opening in the garment may be re-sealed. A continuous and re-sealable seam 19 is desirable from the perspective of thermal insulation, whereas a discontinuous seam may be preferred as it allows intermittent access to the patient's limbs without the requirement to modify the garment in situe in the garment 10. Alternatively, the seam 19 represents a weak spot which can be ripped or torn to facilitate removal. It will be understood that either manifestation of the invention is within the scope of the instant device 7.

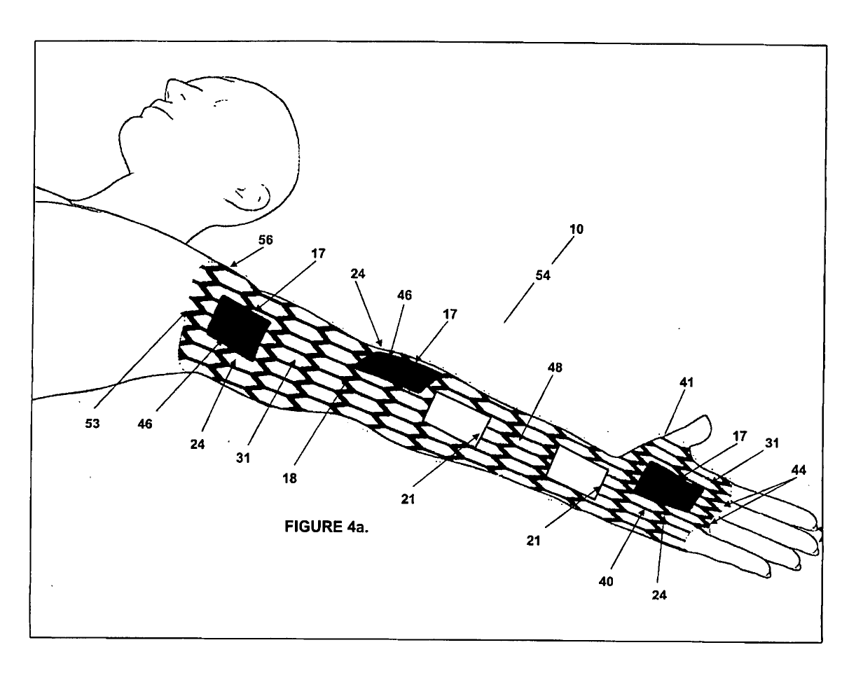

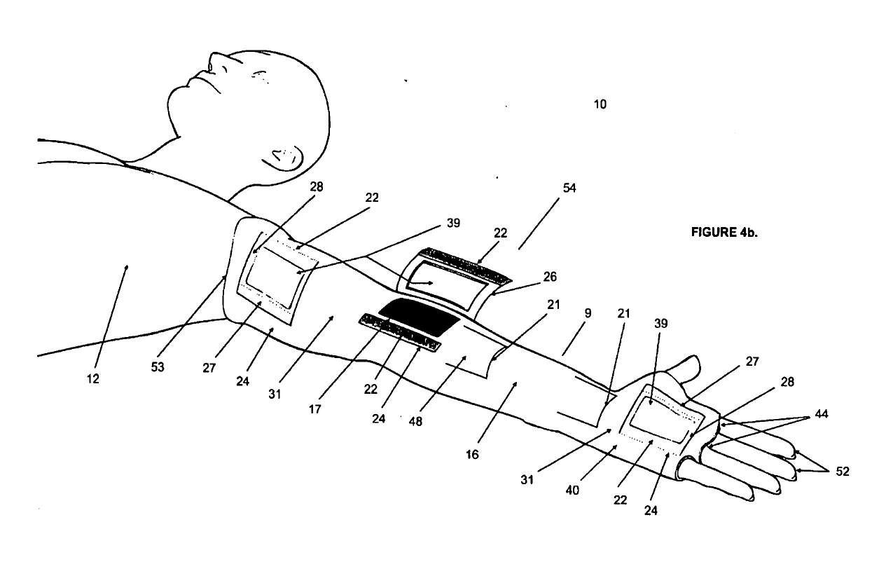

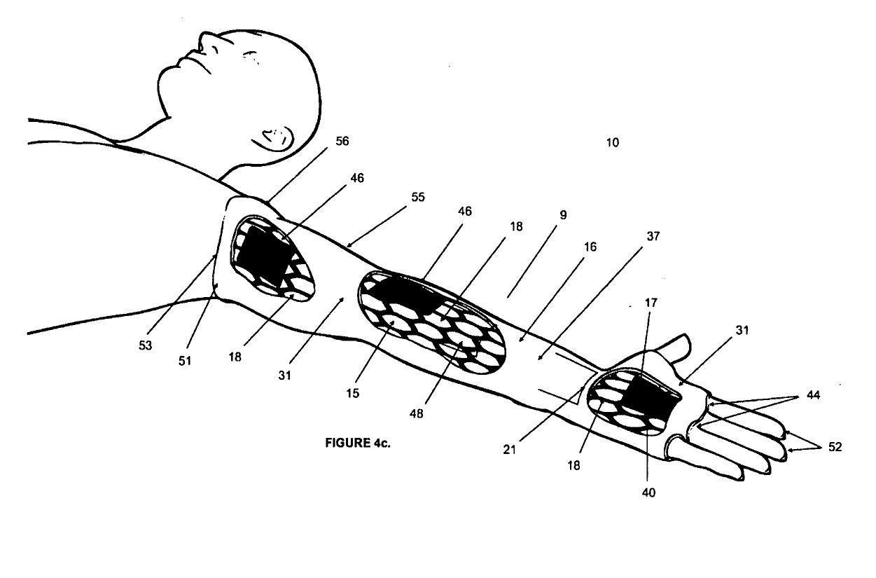

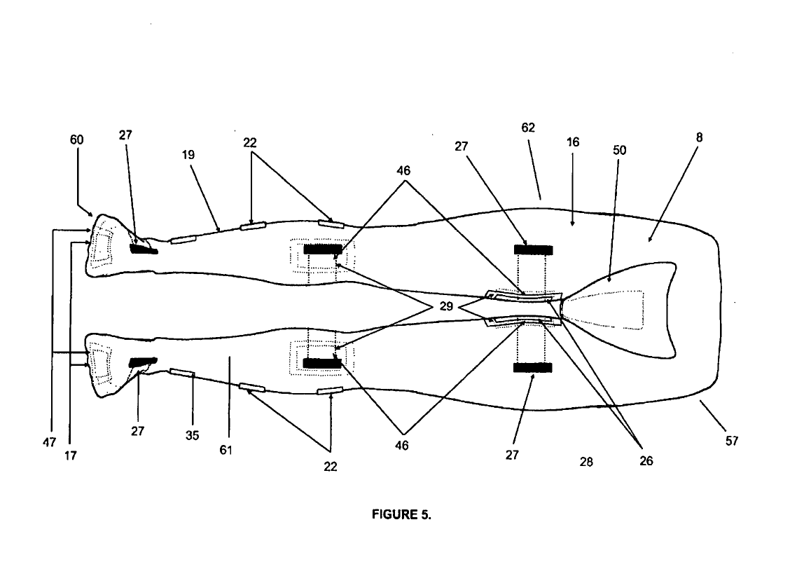

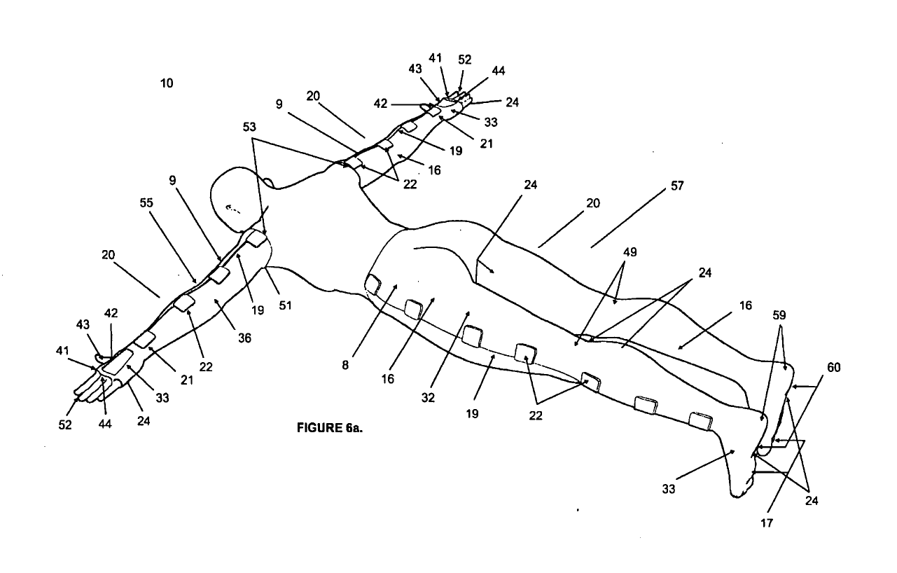

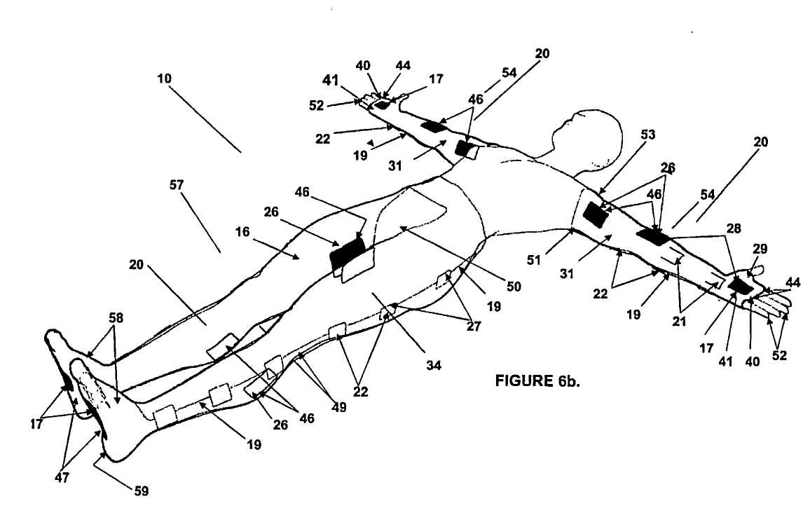

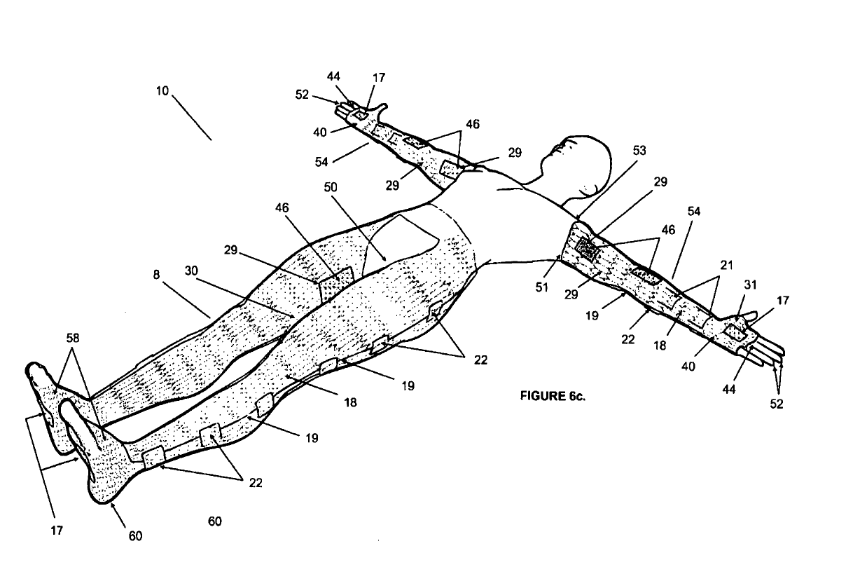

Seams and openings 19 in the instant device 7 may also be used to increase the utility of the instant invention 10. During surgical intervention it may become, for example, necessary to insert a cannula into a vein. Taking as a preferred example, cannulization of the ante-cubital vein, the re-sealable element 19 may comprise a circumferential cut 21—FIG. 4a, 4b and FIG. 6—preferentially on the inner face of the elbow section, which allows for the insertion of the cannula or any other attachments as may be desired. Once the intervention has occurred, the un-sealed element may be partially re-sealed to prevent unwanted heat-loss. The re-sealable seam element 19 may be configured as an overlapping fabric section, a hook-and-loop attachment, as illustrated, 22 or as a Ziploc® type feature or any other re-sealable means which has the desired utility. In an alternate configuration the seams or openings 21 may run circumferentially around the limb at specific intervals 21 in order to allow access at points of site specific surgical interest such as wrist, elbows, the knees and the thighs, whereby instrumentation may be attached directly to the skin or intra-vascular cannulas inserted.

In yet another alternate configuration the seam may comprise single-use perforations which are not re-attachable.

Incorporated either within the fabric of the inner material layer 15, or mounted on the outer surface 23 of the inner material layer 15 which is proximate the patient's skin 12 are a series of thermally conductive filaments 18. The filaments 18 may be arranged to run co-axially 24 with the principal axis of the limb 20, in a web like network, to follow the principal axes of the venous or arterial routes, or in any other manner which promotes effective heat transfer from the exothermic heat element 17 to the outer surface of the inner layer 23 of the material 15 which encloses the patient's limbs 20. Therefore, the filaments 18 may be configured either continuously or discontinuously configured longitudinally, axially, or along arterial or venous pathways or any advantageous combination thereof.

The filaments 18 may assume any appropriate dimensions with variations in, length, thickness, form, inter-connectivity or shape which are conducive to heat transfer. In addition, the network filament properties should be selected for their properties of thermal conductivity, which properties may be varied both by thermal conductivity and by surface area to impart greatest benefit to the patient. The arrangement of the heat-conductive filaments 18 should, by design, overlay the areas of the patient's dermis 12 which have the greatest effective heat transfer.

In addition, the heat transfer filaments 18 may be disposed about the inner layer 15 of the device 10, in any manner which is conducive to effective heat transfer; by surface mounted adhesive means, by weaving or by the embedding of discontinuous metal fragments or filaments, within the body of the substrate fabric 15 or by any other means. As an example, one material which may be utilized for the effective transference of heat is Kapton® tape which has a coefficient of thermal conductivity of 0.60 W/mK. This material has the benefit of being relatively thin and flexible and is well known in the electronics industry for being able to be pre-printed and die-stamped in an economically advantageous manner. This facilitates the creation of a web-like thermal dispersive network 18 which is capable of a high degree of conformance to a variable three-dimensional form such as the human limb. In addition it can be created in pre-printed and pre-stamped formats, applied directly to the inner fabric substrate 15, which will significantly improve the economics of the invention. Other materials which may be considered which have significantly higher thermal conductivity coefficients than Kapton® tape are aluminized Mylar® tape or C695 non-electrically thermal insulating tape, manufactured by Saint Gobain and which has a thermal conductivity of 2.0 W/mK. A combination of materials which have varying thermal coefficient properties may also be advantageously utilized. Evidently a wide range of materials may also be used for the purposes of effective heat transmission, without departing from the spirit or scope of the invention.

Turning now to the heat element 17; the preferred method of providing a heat source to the patient's limbs is via an exothermic heat pack 17 as previously described herein. Although it is within the scope of the instant invention to have the heat-pack 17 placed adjacent the patient's skin 12, the inventors consider that this compromises the efficiency, effectiveness and versatility of the instant method and device 10. As the preferred heating mode is derived from an air-activated exothermic chemical source 17, the reaction can be controlled through the amount of air which the heat element 17 is allowed to absorb. However, the inventors assert that placing the heat element 17 adjacent to the patient's skin 12 places an upper temperature constraint on the exothermic reaction without yielding preferential advantage which may result in inadequate thermal reaction and compromised heat distribution. Alternatively, the heat element 17 may produce undesirably high levels of heat which may cause the patient harm if placed in direct contact with the patient's dermis 12 for protracted periods of time.

Therefore, preferentially, the heat element 17 is situated on the exterior surface 23 of the garment's inner layer 15 and advantageously positioned to distribute heat to areas of the dermis which have greater heat absorption capability 24 as illustrated in FIGS. 4a, 4b and 6a. Preferentially, the heat pack 17 may have its interior face resting on the web of thermally conductive filaments 18 so as to transfer heat from the area immediately beneath the heat pack 17 and increase the actively heated proportion of the patient's limb 20.

The heat pack 17 is preferentially of the type which is exothermically activated on contact with air, as, for example, that disclosed by Yim, U.S. Pat. No. 6,886,553, being a mixture of, “approximately 35-50% by weight of iron powder, 25-45% by weight of water, approximately 10-14% by weight of water retaining agent and approximately 4.5-6% by weight of salt.” Upon exposure to the air oxidation of the iron begins an exothermic reaction. Yim further provides guidelines for the range of heat radiating from the apparatus, defining it as between 39° C. and 45° C. and provides the rationale for this temperature regime as “in order to provide a level of heat suitable for therapeutic heating without danger of burn to human skin.” Yim further defines the heat emission range of “between 39° C. and 45° C. for approximately 12 to 18 hours.”

As the heat pack 17 is activated by exposing the core mixture to air, Yim confirms that the amount of air which is allowed to reach the mixture creates a proportional exothermic reaction which contributes to an understanding of the relationship of thermal output to heat-pack longevity. It is the inventors' intention to advantageously deploy heat packs 17 which are re-sealable such that a partial or total re-sealing of the heat element 17 results in a reduction or extinction of heat output. The degree of variability which this feature confers improves exothermic flexibility which results in greater utility in the operating room.

Notwithstanding the inventors' preferred intention to preferentially utilize exothermic air reactive heat elements it is within the scope of the invention to utilize any other self-contained heating elements. These heat elements may be single-use and disposable or multi-use and reusable, without departing from the spirit of the instant device.

Considering now the preferred method of deploying the heat element 17; prior to activation the heat pack 17 is a sealed element. For air activated exothermic heat packs 17 as described above, the integrity of the airtight seal 25 has to be maintained in order to prevent unwanted activation of the single use heat element 17. For this reason, the inventors conceive that the heat element 17 is packaged and transported with an external cover 26. The cover 26 prevents both accidental transit damage to the heat pack 17 and also undesirable patient interference in the pre-operative environment. The heat pack cover 17 may beneficially be hinged to the outer surface 27 of the inner layer of material 15. The heat pack 17 is equipped with an air-tight seal 25 (FIG. 2b, only), which may be disclosed by folding back the protective cover 26. Once the air tight seal 25 is breached, a vent opening 28 is created, the exothermic reaction begins and the heat pack 17 creates heat. As the temperature generated by the heat pack 17 is derived from a chemical reaction which is proportional to the amount of air which is allowed to access the chemical mixture contained therein, the heat pack 17 may, advantageously be equipped with a plurality of vents 28 (only one is illustrated) with which to control the chemical reaction and thus the heat output.

The heat pack 17 may either be configured as an integral element of the garment 10 or may be configured to be positioned inside a pocket 29 on the garment; it may be detachable, or be permanently attached to a surface of the garment 10 without departing from the spirit of the invention. In an alternate embodiment the heat pack 17 can be added to the garment 10 after the patient is clothed in the garment 10.

Referring now to FIGS. 4 through to 6, in order to create the greatest versatility, the garment 10 may be equipped with multiple integral heat packs, 17 which may be surface mounted or detachable or heat pack enclosures or pockets 29 which are preferably configured to apply heat to the medial face of the leg 30 and the ventral side of the arm 31. Clearly the number and position of the heat packs 17 as deployed and activated is determined by and dependent on the individual patient's specific heat requirements.

For the additional safety and protection of the patient, and as a means of informing the anesthesiologist about the patient's skin temperature, the external surface 32 of the outer layer of the garment 16 may incorporate or be coated with a surface layer 33 which has been painted, printed or treated with thermal ink 33. Thermal ink 33 is well known as a means of monitoring temperature and the inclusion of a thermal indicator in the garment 10 serves two purposes: it indicates to the surgical staff that heat is being generated effectively and may also serve as an over-temperature indicator in the event that the skin 12 is exposed to temperatures in excess of 42° C. In order for the ink treated area 33 to accurately reflect the actual output temperature of the heat pack as delivered to the patient's skin 12, the garment 10 is configured such that a thermal bridge 34 is constructed between the heat pack and is extended such that it is also in contact with the heat responsive ink coated area 33. Logically the heat conductive filament 18 may be extended circumferentially so that the heat sensitive ink portion is visible on the lateral element 35 of the leg garment 8 or the radial aspect 36 of the garment clothing the arm 9.

In an alternative and preferred configuration, the thermal bridge 34 is constructed between the surface area adjacent to the skin 12 and the thermally active inked area 33 of the garment 10, such that the thermal ink 33 indicates the temperature as delivered to the patient, rather than the thermal source 17 output temperature. Thermal inks 33 are well known in many industries. Thermally reversible chromic inks such as those created by Colour Thermal inks, for example, may have a tolerance of 2% on temperature ranges between 30° C. and 80° C. This results in a temperature indicator scale of 1° C., which is appropriate for the purposes of establishing appropriate temperature measurements for the device 10. Additionally an outer material layer 16 is configured about the external surface 23 of the inner layer 15 of the device 10 in order to preferentially direct the temperature towards the patient's skin 12 rather than allowing unwanted thermal egress into the operating room.

The outer surface of the invention 16 may be formed continuously or discontinuously in order to contain the heat energy and direct it to the patient's limbs 20. In one configuration the outer layer 16 comprises a second fabric layer 37 substantially continuous and conforming to the size and format of the inner layer 15. In an alternate configuration, the insulative cover 16 may merely cover the network of conductive heat elements 18, at specified points, 38 making for a lighter garment 10 construction without substantially reducing the thermal effectiveness of the device 10.

As the preferred heating method is by exothermic chemical reaction, at least a portion of the outer layer 16 which substantially covers the heat element 17 is constructed to allow oxygen to enter the chemical heat pack 17. Such a breathable outer layer 39 may cover a size substantially equal to or larger than a heat pack 17 and may simultaneously display insulative properties which act to diffuse the temperature by containing the heat between the cover 16 and the fabric of the garment 11, thereby allowing the heat to dissipate across a larger surface area than is covered by the heat element itself.

Prior art in the field of provision of heat to the hands, when deployed in glove format, sought to preserve tactile sensitivity which precluded inclusion of a heat pack in the palm area of the glove. In the surgical environment, there is no detriment to temporary removal of feeling from the palm area, which therefore allows for inclusion of the heat pack in the palm of a glove.

The instant device 7, by utilizing the palm area 40 of the glove element 41 of the device 10 to introduce heat to the body, takes preferential advantage of the position of arteriovenous anastomoses located in the palm of the hand 40. Arteriovenous anastomoses, or “AVA's”, are areas of the body where a nexus of veins and arteries are proximate to the surface of the skin 12. These areas have been shown to have significant influence over body temperature. An experiment which confirms this attribute, entitled “Heat extraction through the palm of one hand improves aerobic exercise endurance in a hot environment”, was conducted by Dennis Grahn and Craig Heller at Stanford University and reported in the Journal of Applied Physiology (September, 2005) summarized “The heat extraction technology takes advantage of adaptations for heat transfer that are features of certain non-hairy skin surfaces. The arteriovenous Anastomoses (AVAs) and venous plexuses in the palms of the hands and the soles of the feet are effective mechanisms for heat dissipation when core body temperature rises.”

Although Grahn et al's study was used to quantify improvements to the subject's endurance as a result of direct application of cooling to the palm of the hand, it is the intention of the instant invention to advantageously use the same zonal heat transfer attributes of skin and blood vessels in order to maintain normalcy of core temperature in the patient's peri-operative condition.

The instant invention claims the benefit of inclusion of the heat pack 17 in the palm of the hand 40 as, advantageously, this allows the inclusion of one or more openings 42 in the area adjacent the dorsum of the hand 43 through which to insert a cannula. In addition, the glove 41 may be equipped with a finger opening or multiple finger openings 44 through which to attach a pulse-oximetry device. In fact the glove element 41 of the instant invention 7 may advantageously take the form of fingerless gloves 44.

For the upper extremities 45, preferentially, the glove elements 41 may be elongated proximally, providing passive insulation to the arms 9. In addition to using AVA's located in the palm of the hand 40, the instant device 10 may, without departing from the scope of the instant invention, also be equipped with supplemental heat sources 17 located in alternative locations 46 on the patient's body, which positions preferentially have thinner layers of skin and fat. Typically these zones are located on areas which are free from hair, where skin and fat layers do not act to provide insulation to the circulation system. Such locations include the soles of the feet 47 and may include the ante-cubital fossa 48, popliteal fossa 49, femoral triangle 50, and axilla 51. Each of these areas is advantageous because the circulatory system is in close proximity to the surface of the skin.

Supplementary heat packs 17 may be also be advantageously deployed in the case where the patient's circulatory system has already been compromised, either prior to or during surgery, thus acting to increase the temperature of the extremities, and acting as a means of recovering compromised core temperature.

In an alternative surgical scenario, the heat packs 17 may be deployed sequentially to augment the patient's core temperature in cases where lengthy surgical procedures are envisaged. In this embodiment the patient's core temperature is monitored and additional heat sources 17 are bought on line as required. Practically, in the surgical environment, it may be envisaged that additional heat sources 17 are bought on-line as required in response to reductions in the patient's core temperature.

Therefore, preferentially, the entirety of the limbs 20 may be totally enclosed within the garment 10 and heat applied thereto. Alternatively, for relatively short surgical procedures, or where surgery is planned on the arm 9 or thigh 8, only the forearm and leg may be enclosed. Clearly, coverage of any combination of limbs 9, 8, including total or partial coverage of each limb 10 may be made in order to accommodate patient or surgical requirements without departing from the spirit of the invention.

In the embodiment with the smallest surface area, as previously disclosed, the device 10 when configured for upper body usage is a glove 41. The glove 41 may have variable lengths and may cover from the fingertips 52 to the axilla 51. In its basic form the glove 41 has a heat pack 17 preferentially located and integrated into the ventral structure of the glove 41 covering the palm 40 of the hand. In alternative versions of the invention, additional or supplemental heat packs 17 may be deployed in a variety of alternative locations, 47, 48, 49, 50, 51. These locations are advantageously selected to benefit from areas of skin which have high heat transfer coefficients.

Essentially the device 10 may consist of a tubiform garment which in one version is adjustable by means of Velcro straps 22 to allow easy application as well as facilitating rapid removal in the event of the necessity of emergency access. In an alternative embodiment the tubiform garment may be circumferentially elasticized or equipped with an elasticized gusset (not illustrated) and disposed about the limbs 20 in such a manner as to advantageously provide a degree of compression to the limbs 20. The degree of compression may vary between extremely light compression set—sufficient to retain the garment on the wearer without additional retaining methods and a higher degree of compression designed to assist in combating deep vein thrombosis (“DVT”)

Generally, the garment 10 used to enclose the arm 9 takes a sleeve 54 format. The construction of such structures has been discussed elsewhere and need not be examined in more detail here. The proximal end of the sleeve 54 may be elasticized 53 to fit to the upper extent of the brachium 55, such that the garment 10 may be retained without recourse to additional support mechanisms, although the degree of elastication 53 should be closely monitored so as to avoid unwanted vaso-constriction as a result of limb 20 compression. As an alternative, the garment 10 may be configured to fit over the shoulder 56, thereby obviating the requirement for elastication 53.

From both the perspective of simplicity of construction and also from the point of view of the patient clothing themselves in the instant garments 10 in the pre-operative room, it is preferable to construct the garment 10 for each limb 20 in a single construction. However, it will be understood by those versed in the art that each limb 20 may equally be encased in multi-part garments 10, without departing from the spirit of the invention. Although the practical inference of multi-part garments 10 would be that additional assistance from the surgical staff would be required for correct installation on the patient.

The garment 10 which fits the lower extremities 57, may be preferentially configured as a pair of loosely fitting leggings or trousers 8. This garment format promotes patient comfort, both physically and emotionally, and is equipped with features which assist surgical procedural effectiveness and are consistent with the hypothermia preventive objectives of the instant invention.

A detailed description of the leggings 8 may take as its starting point the distal end of the garment 10 which may incorporate socks 58 or fabric boots 58. Without departing from the spirit of the invention the socks 58 may be integrated into the lower limb 57 enclosing device 8 or may be separate therefrom. Arguably, it is preferable for the garments 10 for the lower limbs 57 are configured to have a sock-like element 58 at the distal end. With a pre-formed heel section 59 incorporated into the device 10 will enable the patient to dress themselves while ensuring that the heat pack 17 orientation is adjacent the sole of the foot 47. Equally, the lower limb garment 8 could be constructed separately as a complete sock 58 with toes.

Preferably the plantar face 60 of the sock 58 is equipped with an exothermic heat device 17 which applies heat advantageously to the sole of the foot 47. Additional heat packs 17 may be placed in a variety of positions on the patient's leg 61 and thigh 62 in such a position as to take full advantage of the places where the veins and arteries of the lower limb 61 circulatory system are in close proximity to the patient's skin 12 and or muscle masses in the limb 57. By incorporating the sock 58 into the trouser garment 8, the orientation of the heat elements 17 is confirmed without additional intervention by the surgical staff.

It is equally within the scope of the invention to use a single heat pack 17 per limb 20, or multiple heat packs 17 per limb 20, depending on the operation to be performed, the estimated duration of the operation and the condition of the patient's circulatory system.

In particular, in the case of the lower limbs 57 where the offset temperature differential between the body core temperature 2 and lower limb skin temperature 6 may be excessive, the heat packs 17 can be configured in such a manner as to compensate for or supplement circulatory temperature. That is to say that the heat applied to the limbs 20 is responsible for increasing the core temperature 1 (FIG. 1) by returning pre-heated blood to the body core, thereby obviating the onset of hypothermia and simultaneously improving body core temperature 1, with all the physiological benefits which this confers.

Therefore, in yet another configuration of the instant invention which may be deployed either independently or in conjunction with the tubiform surgical garments deployed on the upper limbs, the heating device 17 may be configured as an element of a surgical stocking for encasing the lower limbs 57. This configuration is doubly advantageous as it assists in improving lower limb temperature 5 and if it is deployed in conjunction with an elasticized tubiform garment will coincidentally work to counter deep-vein-thrombosis (DVT). Additionally the garments 10 may be deployed and work in conjunction with Intermittent Compression Device (ICD). Practically it may be advisable to commence the surgical operation with a single heat element 17 and then bring additional heat packs 17 on-line sequentially, in response to observed body temperature fluctuations.

In an alternative operational practice it may be advisable to bring all heat packs 17 on-line at the commencement of the operation and then re-seal 25, or partially re-seal the exothermic elements 17 to reduce their thermal outputs as the patient's body temperature stabilizes throughout the operation.

For patients undergoing shorter surgeries it may be preferable to allow some of the heat to dissipate to the environment, rather than confining it in proximity to the patient's dermis 12. In this circumstance the cover 26 may be removed partially or in its entirety or be replaced by a cover 26 which is smaller than the heat pack 17 without departing from the spirit of the invention. However, releasing heat to the surgical environment is generally regarded as detrimental to the wellbeing of the surgical staff. It is the inventors' intention to create a system of immense versatility through the use of adaptive garments 10 with multiple heat pack 17 deployment and positioning options and additionally, multiple modes of operating each individual heat element 17

As previously described in the detailed description of the drawings, the garment 10 may be equipped with a variety of openings which enhance garment utility in the surgical environment.

Indeed it may be possible to construct an index which would suggest to surgical staff the patient's intra-surgical heat requirement. This practical index might be based, upon a patient's overall health taken in conjunction with the ambient air temperature at the point of surgery to arrive at a calculated figure for thermal heat loss. Such an index might take into account among other criteria, the patient's age, body mass, the condition of the patient's circulatory system, the area being operated on, and the intended duration of the proposed surgical procedure, and other factors in order to determine the anticipated likelihood of hypothermia and therefore the number, size and most advantageous network construction of the heating devices 10 For example, in surgery of the upper torso, it may be determined that as this entire area must be uncovered, the heat loss level which this represents will require the instant device 10 to be applied to each of the four limbs 20 in order to maintain normothermic temperature. Correspondingly, for surgery which requires only partial exposure of the torso, fewer garments may be deployed.

We claim:

1. A method of maintaining advantageously stabilized homeostatic parameters through normothermic maintenance in particular in the peri-surgical environment. In particular, the method of advantageously applying an insulative garment over at least one favorably heat receptive area of the BSA in order to counter environmentally induced hypothermia;
the further method of maintaining normothermia using a disposable or re-usable, un-tethered, anatomically formed or tubiform garment or plurality of garments preferentially configured to enclose the extremities and in particular the areas of the body which are the most effective loci of heat transfer;
the further method of incorporating active, un-tethered, self-contained heating means as an element of the garment, said thermal means being linked to simple visual diagnostic indicators showing garment functionality.

2. The method of claim 1 whereby antibiotic prophylaxis is avoided.

3. The further method of claim 1 whereby the positive heating attributes of the system may be augmented through the use of additional insulative garments, which garments may be disposed about the patient's body to achieve greatest benefit to both patient and surgical field.

4. The further method of claim 1, of incorporating leg pumps for inhibiting the pooling of blood and subsequent clots in the extremities of the patient.

5. A fabric garment apparatus, substantially designed to conform around the limbs of the patient undergoing treatment, said garment providing passive insulative means to protect the extremities of the patient undergoing treatment against heat loss; the garment further having integrated un-tethered and at least one self-contained heat element means for arresting patient heat loss and promotion of patient euthermia.

4. The apparatus of claim 5 where the garment is disposable

5. The apparatus of claim 5 where the garment is recyclable

6. The apparatus of claim 5 where the garment is, after cleaning, re-usable.

7. The apparatus of claim 5 where the garment fabric is (structurally) substantially formed as a single, integrated structure

8. The apparatus of claim 5 whereby the form of the tubiform garment may consist of multi-layer fabric integrating passive insulative and active thermally conductive elements therein

9. The apparatus of claim 5 where heat distribution is enhanced through the use of heat conductive tracers, filaments or conduits.

10. The apparatus of claim 5 for deployment on the upper body taking the form of fabric gloves, mitts, gloves with extended cuffs, sleeves or full-arm length gauntlets.

11. The apparatus of claim 5 for deployment on the lower body and taking the form of bootees, slippers, socks, stockings or greaves of variable length;

12. The apparatus of claim 5 where the heat element is one of a range selected from chemical exothermic reaction heaters, gel heat packs, or other heating means which is self-contained and does not require an external umbilical in order to function;

13. The apparatus of claim 5 whereby the heat element is incorporated within the garment, where said element is preferentially of a class which is activated by the removal of a seal and exposure of the heat element to the atmosphere.

14. The apparatus of claim 5 whereby the heat element may be positioned inside a pocket on the garment.

15. The apparatus of claim 5 whereby the heat element is detachable from the garment.

16. The apparatus of claim 5 where the heat pack is added to the garment after the patient is clothed in the garment.

17. The apparatus of claim 5 where thermal indicator means may be incorporated within the garment structure, allowing instantaneous visual indications of garment thermal output.

18. The apparatus of claim 5 within which may be integrated access means for purposes of carrying out medical procedures including, for example; pulse oximetry, cannulization, and intravenous drips.

19. The apparatus of claim 5 which is equipped with a detachable seam for removal of said garment.

20. The apparatus of claim 5 where the garment fabric is (structurally) substantially laminate in format, comprising a (passive) lightly insulative inner layer, an active exothermic intermediate layer and an external, (passive) more heavily insulative layer wherein a network of heat distributive filaments is deployed within one or more of the layers, as required.
